(19) United States
(12) Patent Application Publication
(10) Pub. No.: US 2022/0054043 A1
(43) Pub. Date: Feb. 24, 2022

(54) METHOD FOR EVALUATING USAGE DATA

(71) Applicant: Otto Bock Healthcare Products GmbH, Wien (AT)

(72) Inventors: Erik Albrecht-Laatsch, Rosdorf (DE); Alexander Pappe, Wien (AT); Dirk Seifert, Wien (AT); Alexander Glier, Göttingen (DE); Philipp Kampas, Wien (AT); Christian Hofer, Wien (AT); Mario Koppe, Göttingen (DE); Christopher Bartels-Sellering, Göttingen (DE); Jens Drieseberg, Clausthal-Zellerfeld (DE); Martin Schmidt, Duderstadt (DE); Robert Kaitan, Wien (AT); Markus Schachinger, Wien (AT); Juan Pablo MEJIA NINO, Mödling (AT); Sven Faulhaber, Seulingen (DE); Jörg Krukenberg, Duderstadt (DE); Katharina Olze, Braunschweig (DE); Andreas Eichler, Wien (AT); Florian Gishamer, Wien (AT); Bernhard Prochaska, Wien (AT); Christian Kovacs, Wien (AT); Eva-Maria Fink, Wien (AT); Gregory Lyle Schneider, Salt Lake City, UT (US); Sebastian Amsüss, Wien (AT)

(73) Assignee: Otto Bock Healthcare Products GmbH, Wien (AT)

(21) Appl. No.: 17/416,431

(22) PCT Filed: Dec. 19, 2019
(86) PCT No.: PCT/EP2019/086267

§ 371 Date: Jun. 18, 2021

(30) Foreign Application Priority Data
Dec. 20, 2018 (DE) 10 2018 133 078.2

(51) Int. Cl. A61B 5/11; G16H 10/20; A61B 5/00

(52) U.S. Cl. CPC A61B 5/112; G16H 10/20; A61B 2562/0219; A61B 5/1122; A61B 5/6812

(57) ABSTRACT

The invention relates to a method for evaluating usage data of at least one orthopaedic device which is equipped with sensors for detecting properties, states, or changes in properties or states, wherein the sensors are connected to a transmitter directly or via a storage device and the transmitter transmits the sensor data provided by the sensors to an evaluation unit in a computer network in which the sensor data is processed.

The invention relates to a method for evaluating usage data of at least one orthopedic device, which will be equipped or is equipped with sensors for detecting properties, states, or changes of properties or states of the orthopedic device or of the user of the orthopedic device.

Orthopedic devices such as orthoses, prostheses, or also collars, wheelchairs, exoskeletons, and the like are individually used. Orthopedic devices can include drives, blocks, brakes, and/or dampers which are activated or deactivated via a corresponding control unit. The control unit can be connected to one or more sensors in order to change the behavior in the orthopedic device on the basis of sensor data.

A device and a method for remote maintenance of an electronically controllable prosthesis are known from U.S. Pat. No. 6,679,920 B2. The prosthesis is equipped as a prosthetic leg with a prosthetic socket, a prosthetic knee joint, a lower leg part, and a prosthetic foot. A hydraulic damper is arranged between the prosthetic socket and the lower leg part, which adjusts the damping of the hydraulic damper on the basis, for example, of forces or accelerations. A separate maintenance device or docking station can be connected to the control unit of the prosthesis. The maintenance device collects the control and movement data of the prosthesis, assesses them, processes them, and if necessary supplies the control unit of the prosthesis with an updated data set. The data transfer can take place wirelessly via a data network or a telephone line.

U.S. Pat. No.10,015,840 B2 relates to a device and a method in which sensor data of a separate portable unit are collected and compared to a movement model. If the model does not correspond to the sensor data, correction commands are transmitted. As soon as sufficient correction is present, the correction is ended.

Presently, the user of the orthopedic device, a therapist or orthopedic technician, the payer, for example a health insurance company or a care relief organization, or the producer of the orthopedic device do not know whether the user of the orthopedic device uses it completely and correctly and whether it functions correctly. Standardized questionnaires are sent out in the field of prosthetics, filled out by the patient, and clinically validated. Such a data collection method has the disadvantage that it depends on the personal assessment of the user of the prosthesis. The respective perception as to whether the prosthesis is correctly seated, is pleasant to wear, and corresponds to the expectations and requirements, is different from patient to patient, so that a comparable data situation for an objective judgment is not present. The feedback of the user is also rather unreliable in the case of short-term uses, for example in the case of loan equipment.

The object of the present invention is to provide a method using which usage data of an orthopedic device are detected and can be objectively evaluated.

This object is achieved according to the invention by a method having the features of the independent claim, advantageous embodiments and refinements of the invention are disclosed in the dependent claims, the description, and the figures.

The method for evaluating usage data of at least one orthopedic device, which is equipped with sensors for acquiring properties, states, or changes of properties and states, wherein the sensors are connected to a transmitter directly or via a storage unit, provides that the transmitter transmits the sensor data provided by the sensors to an evaluation unit in a computer network, in which the sensor data are processed and evaluated. The orthopedic device is in particular designed as a prosthesis or orthosis of the upper or lower extremity or as a mechatronic system, for example as an exoskeleton or a similar device, which is operated together with a user or patient. The sensors are preferably located in or on the orthopedic device and acquire properties, states, or the changes thereof with respect to the orthopedic device and/or with respect to the user of the orthopedic device. The sensor or sensors are either connected directly to a transmitter or are connected via a storage unit to a transmitter in order to transmit the data from the sensor to a device outside the orthopedic device. The transmission of the data can be carried out in a wired manner, by data carrier exchange such as storage card transfer, or via a direct contact. It preferably takes place wirelessly, for example via a Bluetooth connection to a mobile terminal, for example a mobile telephone, a smart watch, a tablet, a mobile computer, or a similar data transfer device, which is connected or can be connected to the orthopedic device. The orthopedic device itself can also include a so-called SIM card and/or a mobile telephone module, so that it is possible to transmit the data directly into a computer network from the orthopedic device. The computer network can be designed as a so-called cloud and can have a decentralized structure. It is thus possible to enable a decentralized access to the computer network. In addition, hardware and software resources are better utilized. The evaluation unit is implemented in the computer network, for example in the form of an analysis program which is coupled to a database in the computer network in order to store and evaluate the usage data and user data therein.

Movement data, operating data, and/or loads of the orthopedic devices or sensor data about the user of the orthopedic device are advantageously detected by the sensors and transmitted to the evaluation unit. The movement data and operating data are, for example, velocities, duration of the movement, frequency of movements, movement patterns, repeating movement patterns, forces, torques, accelerations, orientations in space and relative to one another if multiple components of an orthopedic device are coupled to one sensor or multiple sensors. Data about the user are also relevant, for example, weight, height, body temperature, pulse, respiratory frequency, or the like. The data are retrieved and transmitted, for example, upon request from the user himself, an orthopedic technician or a therapist, a physician, the producer, or a care facility, for example a health insurance company or the like. The user can also be requested to transmit, for example via the evaluation unit, which can be equipped with a request function. Alternatively and additionally, the data are permanently transferred in real time during the use. The data transfer on request can take place after reaching a specific limiting value, for example a chronological limiting value, a load limiting value, after reaching a specific repetition number or after occurrence of a specific event, for example a peak beyond a predetermined limit load or a detected accident. An important check in the case of noncontinuous data transfer is also how the load has taken place. There is the possibility of reaching limiting values with a single maximum load or via a plurality of sub-threshold loads. The respective values can be reached in a few days with intensive use or via a regular use over a longer time period. Relevant parameters for this purpose are, for example, the frequency of wearing the orthopedic device, for example the leg prostheses, how many steps are executed, which movements and/or exercises are carried out, whether the user fulfills the exercise target placed by a therapist, and the like. A combination with data from smart devices such as smart watches or the like is also conceivable for ascertaining these data.

The sensor data are preferably acquired and evaluated over the entire usage time period of the orthopedic device or the orthopedic devices, whereby it is possible to acquire a complete chronological sequence of the use and to evaluate the usage data over the entire usage time period. Alternatively, the sensor data of one or more orthopedic devices are acquired and evaluated in a chronologically limited manner. The duration and the beginning and the end of a respective data acquisition and evaluation and the identification of when which orthopedic device is the subject of the data acquisition are documented and associated with the respective data set. It can thus be documented who has used the orthopedic device when and how. In the case of an orthopedic device attachable to the body of a patient, a type of logbook in digital form can be created, in which all data and settings of the orthopedic device and about its use are stored. Data about the user of the orthopedic device can also be acquired and evaluated and also stored. The data of the orthopedic device are advantageously personalized and associated with the respective user, in order to be able to make statements during the evaluation and after the evaluation of the sensor data as to whether the orthopedic device and the user are optimally adapted to one another or whether another orthopedic device would match better with the user or whether the orthopedic device has to be adapted or changed.

The sensor data which are stored in a database and evaluated in the evaluation unit can be supplemented by assessments of the user of the orthopedic device and/or third parties. The assessments can be carried out, for example, recorded on the basis of questionnaires, which are transmitted to the user or other persons. The questions can be placed online and answered online, for example via the respective terminal via which the user or a third party comes into contact with and is connected to the computer network. For example, at regular intervals the user and/or a third person, for example an orthopedic technician, a therapist, a mechanic, or another person from the environment of the user of the orthopedic device, can be asked which impressions the respective person has of the usage and of the orthopedic device. The objective sensor data are thus supplemented by subjective data, whereby it is possible to correlate the recorded data with subjective perceptions. It is thus possible that the orthopedic device is improved, in particular producers of the orthopedic device who have access to the evaluated data and the assessments can make inferences about the acceptance of individual measures and features of the orthopedic device. Alternatively to the assessments on the basis of provided questions, these assessments can also be given freely. The evaluation unit evaluates the assessments or remarks on the orthopedic device in the scope of the text analysis and associates the assessments of the respective orthopedic device with a specific behavior, a specific component, and/or an item of software which is responsible for the control of the orthopedic device.

The sensor data can be transmitted, as already stated, in real time and also over the entire usage time. Not all data always have to be transmitted, rather only changes or deviations from an expected behavior can also be transferred as data. If a storage unit is present on or inside the orthopedic device, a transfer can in particular take place during the usage pauses of the orthopedic device, for example in the evening after a prosthesis or orthosis has been taken off or when it is detected that no movement or no usage of the orthopedic device is present. Alternatively or additionally, a data transmission can take place at fixed times, i.e., at regular intervals, on request of the evaluation unit or third parties, or by active transmission by the user.

To prevent a data misuse, the sensor data can be coded in a personalized and/or device-specific manner. In addition, the data can be encrypted so that only a limited circle of persons can obtain access to the data. The data can also be anonymized to be able to allow third parties access to the findings and results in the context of medical studies or to refine the orthopedic device.

In particular if a plurality of users of an orthopedic device, for example all users of an orthopedic device of a certain type or a certain model, are connected to the evaluation unit, informative analyses can be created about the usage behavior and the quality and functionality of the orthopedic device and substantiated findings can be obtained.

One refinement of the invention provides that the sensor data of various equivalent or comparable orthopedic devices are compiled and evaluated in the evaluation unit. Due to the compilation or clustering of sensor data and/or assessments of users or third parties on specific models, versions of models, software versions, or equipment variants, improvements or problems can be recognized rapidly and with high reliability due to the broad database. Benchmarking of the users or operators can also take place within application clusters, which can in turn be used as an incentive or motivation aid via a competitive concept and can contribute to accelerated therapy success.

One refinement of the invention provides that a risk analysis is carried out on the basis of the sensor data, wherein a recommendation to the user, a payer, an orthopedic technician, a therapist, and/or a producer of the orthopedic device is output on the basis of the results of the risk analysis. The risk analysis includes, for example, statements about a possible failure probability, a statement about possible wear states, about a malfunction due to a confluence of unpredicted events, which were not yet known upon the creation of, for example, an item of software for the control of the orthopedic device. It is thus possible to recall devices early, to warn users or third parties, to take safety precautions, to perform automatic updates and safety measures, for example by a remote transfer of new programs, or to cause similar things in order to protect the user or third parties from consequences of a failure or an incorrect operation.

One refinement of the invention provides that usage instructions, warning messages, and/or requests are automatically communicated to the user on the basis of the evaluation in order to improve the usage behavior. For example, the user can be given the instruction of how he can better use the orthopedic device and/or how he can improve his physical condition, for example by a transmitted training program or by representations of how the orthopedic device can or should be set, adjusted, or manipulated or used in other ways. Furthermore, warning messages about potential incorrect uses, detected incorrect uses, or maintenance intervals to be observed or not observed can be communicated. The communication takes place via the computer network and a receiving unit, which is arranged in the transmitter unit or in the mobile terminal, for example the smart watch, the mobile telephone, a tablet, or a similar receiving and playback unit.

The orthopedic device can be provided with a control unit or associated with a control unit, which is in turn coupled to a receiver, via which control data, access rights, sensor calibrations and/or changes and/or recordings about changes are transmitted. Furthermore, it is possible to carry out remote maintenance, to ascertain changes due to a special usage behavior on request or automatically, and to detect and compensate for wear affects in that sensor calibrations are performed or a damping behavior is varied.

One refinement of the invention provides that settings, changes of settings, calibrations, accesses to the sensor data, and access rights are stored and kept retrievable in the computer network, in order to be able to document and archive the collected data from the sensors and from the users or from third parties about the assessments or commentaries over the usage duration. A type of time machine is thus provided, by which it can be tracked when someone and possibly who has performed, changed, or used which settings, how the orthopedic device was used, which problems occurred, or which settings and adjustments were particularly advantageous or disadvantageous. This can be implemented, for example, with the aid of a block chain technology. From the data history, it is possible to be able to recognize a development or evolution and possibly to be able to make statements about required maintenance intervals, repairs, changes in the control, or requirements for updates. Resets of the orthopedic device to settings which have proven to be correspondingly advantageous can then also be carried out.

One refinement of the method provides that multiple orthopedic devices are coupled to one another and exchange data via the computer network. The data can be changes in the software, sensor calibrations, or also usage data. The data exchange can be moderated via the evaluation unit and/or the computer network so that not all data of one orthopedic device have to be transferred to the other orthopedic device, but only those data which are relevant for a specific purpose. Advantageously, only identical or equivalent orthopedic devices are coupled to one another via the computer network, since it is to be presumed that identical orthopedic devices have a fundamentally identical task and can require or exchange identical relevant data. However, it is also possible that different orthopedic devices having comparable or technically similar properties are coupled to one another to exchange data and to establish a network with one another.

The data from the computer network and to the computer network are preferably transmitted via a mobile terminal, wherein the data can comprise the sensor data and also the assessments and/or questions as well as instructions, software updates, sensor calibrations, and the like. The mobile terminal as a coupling point to the computer network is connected to the orthopedic device in the respective suitable manner, for example via a so-called Bluetooth connection or another type of data transfer between devices over a short distance via a radio technology. In principle, connection-free and connection-required transfers from the mobile terminal to the orthopedic device and back are possible. A radio connection replaces wired connections between the devices, however, a coupling via wires is also possible and provided in principle.

One refinement of the invention provides that summaries of selected parameters which are acquired by the sensors or are derivable from the sensor data are transmitted to the user of the orthopedic device, a payer, an orthopedic technician or therapist, and/or a producer of the orthopedic device at predetermined times. Via this report service it is possible to reduce the amount of data to be transferred and to already carry out a preselection of the data to be transferred. The selection and possibly calculation or derivation of the parameters from the sensor data can be carried out in the respective orthopedic device in order to utilize the computer capacities present therein, for example, when the orthopedic device is presently not being used. In particular parameter curves, thus the development of parameters over time, can be of particular interest for the corresponding person group or institution, since inferences about usage behavior, technical changes to the orthopedic device, and requirements for adjusting or changing parameters or the orthopedic device can be derived from these curves. If only parameter curves are ascertained and displayed and in particular also transmitted, the amounts of data to be transferred are also reduced.

One refinement of the invention provides that a specific data structure is created with each user and/or for each orthopedic device, on the basis of which the sensor data are selected and transmitted to the evaluation unit. The data structure of the data or assessments to be recorded is individualized, in order to achieve a maximum information capacity from the sensor data, on the one hand, and only have to acquire those sensor data, on the other hand, which are necessary for the care of the user of the orthopedic device or for the optimum use of the orthopedic device. If a user is not capable of executing a specific movement or using the orthopedic device in a specific way, for example, a sensor value which indicates such a use or permits inferences to be drawn about it is not recorded in the evaluation profile and a corresponding field of the data structure remains free.

This also applies to an individual adaptation of an orthopedic device to the respective user, which is accompanied, for example, by changed hardware configurations and/or software configurations. Accordingly, only those data are requested by the specific data structure which are relevant for the respective orthopedic device and/or the user or are implementable by them.

One refinement of the invention provides that a selection from multiple alternative orthopedic devices is made on the basis of the evaluation of the sensor data and/or the assessments and/or data and/or assessments of other users, and this selection is proposed to the user of the orthopedic device, an orthopedic technician, and/or a payer. The selection can be carried out, for example, by a producer or a central evaluation agency and/or the payer, wherein the proposal for an alternative orthopedic device or alternative care can be produced on the basis of the movement data, the usage data, the degree of the utilization of the dynamics of the movements, the movement and usage duration, and/or other parameters. For example, careful handling and regular maintenance can also be proposed as an aspect for the care of a patient having a high-quality orthopedic device. If it should prove that the present care using the orthopedic device is not needed by the user, the user thus does not exhaust or cannot exhaust the possibilities of the orthopedic device or less complex care is sufficient due to changes in the usage behavior, corresponding alternative care or an alternative orthopedic device can be proposed and used for the respective user. As already stated above, alternatively or additionally to an alternative orthopedic device, a training program, an education program, or other instructions or suggestions can be transmitted to the user or a therapist in order to optimally use the presently existing and used orthopedic device and to achieve the maximum utility for the patient.

One refinement of the invention provides that the evaluation unit correlates data about the user of the orthopedic device and/or the orthopedic device and/or comparable orthopedic devices and derives probabilities about malfunction or disadvantageous effects therefrom and transmits these probabilities to the user, the orthopedic technician, the payer, and/or the producer. This enables the timely warning of possible malfunctions, without the malfunction already having occurred. A high accuracy of predictions and probabilities is enabled by the broad database. The broad database is ensured by connecting a plurality of orthopedic devices to the evaluation unit via the computer network.

The sensor data and/or evaluated sensor data are preferably stored in a database, which is optionally secured, so that only authorized persons or devices have access to these data. The data in this database represent a valuable foundation for the refinement of orthopedic devices and improved use by the respective patient. The access to the evaluation unit and/or the computer network can be restricted to maintain data protection guidelines and avoid improper use of data.

The orthopedic device is preferably used as a prosthesis or orthosis, wherein the sensor data are acquired during the use of the orthosis or prosthesis.

An application or app can be installed on a mobile terminal, for example the smart watch, a mobile telephone, a tablet, or the like, using which a connection is established between the orthopedic device and the mobile terminal and in addition between the orthopedic device and the computer network to the evaluation unit. Both the handling of the orthopedic device and also the data transfer are thus simplified. A direct communication between the evaluation unit and the computer network with the orthopedic device can be established by activating the app, without an orthopedic technician or further person having to be interconnected in order to extract data or to transfer data into the orthopedic device or the mobile terminal. Updates can be transferred via the app and the mobile terminal to the orthopedic device without cumbersome setting having to be carried out by the orthopedic technician. Furthermore, a communication can take place between various orthopedic components, various parts or assemblies of an orthosis, a prosthesis, or an exoskeleton, or between the orthopedic component and a smart device, which acquires physical data such as pulse, body temperature, and the like. The smart device can also acquire location data via a GPS system and/or record items of environmental information via a camera and link them to the sensor data, so that environmental influences and ambient conditions can also be incorporated in the judgment of the usage.

A control unit having a CPU or a computer is integrated in the orthopedic device, in order to process the sensor data and to process the orthopedic device in accordance with a control program, which can be stored in the CPU. This computer unit or CPU is advantageously used during the nonusage of the orthopedic device, for example during the charging of a rechargeable battery or during the cleaning, for example at night, in order to execute autoadaptive algorithms, install updates, document changes, or transfer sensor data to the computer network and the evaluation unit.

So as not to restrict the operation of the orthopedic device or overload the computer unit or CPU by the transfer of data, selective or atomic data transfer can take place, in the case of which all data of one sensor or all sensors are not always transferred, but only those data or data fragments which are presently needed. Data preprocessing can already take place inside the orthopedic device, for example in a prosthesis joint or orthosis joint, by way of the computer unit present therein, so that only the changes or the change service are still transferred.

The data transfer can also only take place when the prosthesis is actually used, for example in the context of activity monitoring. The activity data can be acquired as individual incidents or events having qualitative properties. The individual incidents or events are defined, for example, when two sensor variables simultaneously reach a certain limiting value, so that a specific state can be concluded therefrom. This also applies for a data progress. For example, in the case of a prosthetic hand, if the progress from an open hand to a closed hand can be dictated, in the case of which the prosthetic thumb, prosthetic index finger, and prosthetic middle finger press against one another at the end of the movement, this can be classified and stored as a so-called pincer grip. The entire data progress is then defined as a pincer grip and correlated with an incident value or an incident number. Only the number of the pincer grips then still has to be transmitted, without all data about the progress of the movement of the prosthetic hand having to be transferred, without the information content changing for the evaluation unit or the user or another device. This also applies for establishing a force curve in a prosthetic foot, during which first the front foot is loaded, a rear pivot of the lower leg part is detected, and then an increased heel load is recognized. Such a scenario can be identified as walking backward and can accordingly be stored as an individual incident or event. The respective incident or event can be provided stored together with a time stamp and a date stamp and transferred via the transmitter.

The data can be stored in the computer network or in the cloud either in a relational database or multidimensionally. The data are stored in a so-called cube, a cube structure, or a so-called OLAP cube or OLAP data cube. Online analytical processing (OLAP) is possible by way of the data storage in a cube structure, whereby analysis and query can take place from different perspectives and in different levels of detail due to the storage of the data in various dimensions. Evaluations having different degrees of information and different processing complexity are possible due to the selection of a specific perspective or a specific level of detail. Moreover, criteria, on the basis of which the data are evaluated and compared to one another, can be combined with one another nearly arbitrarily. Preferably, in spite of the storage in the complex cube structure, an operating portal for the evaluation of the data is provided, which is particularly user-friendly and enables easy access to the data and an easy ability to link the data among one another. Using such a front end, various users can use the data without programming knowledge, for example the data in the computer network can be made accessible to research and development, service, product design, the therapist, and other users.

The data stored in the database and in the computer network can provide a setting aid, which is transmitted to a mobile terminal, for example, during the setting process of an orthopedic device for adjustment to the respective user. The setting values are compared to typical setting values or to setting values for patients having similar disposition and provided with recommendations, so that the respective user can recognize whether he is located within a setting range, in particular a typical setting range, or an extreme setting has been selected, which is very unusual and is therefore to be checked. The checking and the comparison can take place during the setting process. Thus, for example, all setting processes which have been carried out by greatly varying orthopedic technicians, users, or suppliers can be stored in the database and made accessible for all participants or users of the computer network, possibly in prepared form. A typical setting range for a corresponding component or a corresponding parameter, which can be displayed, for example, on the mobile terminal of the setting person, results from the large number of the recorded, analyzed, and stored data. If the actual setting range is then found, independently of whether it is inside or outside the predetermined or proposed range, the performed settings are stored and transmitted to the computer network or the cloud via the transmitter or the mobile terminal.

The data stored in the computer network can also be used during the production of an orthopedic device, in particular during the production of an individual orthopedic device attachable to a patient, such as a prosthesis or orthosis. For example, a limb or a limb stump can be acquired via a scanning unit, for example a high-resolution optical scanning unit. A digital model of the stump or the limb is generated in a computer or in the computer network from the image data. The data of the limb or the limb stump are compared to already existing data of limbs or limb stumps of other users, checked for common features or differences, and checked as to whether and which embodiment of a prosthesis or orthosis is particularly well suitable or less suitable for the respective user. The data from the computer network, thus the digital experiential data of the other orthosis or prosthesis users or the evaluated data from a plurality of already existing orthoses or prostheses, are incorporated into the manufacturing process. For example, specific material reinforcements or individual components or materials can be used in particularly thick or thin form in various regions to achieve an optimum design of the orthosis or prosthesis. The manufacturing data thus adapted are supplied, for example, to a 3D printer or a similar additive production method, so that the experiential values of the usage data are incorporated directly into the manufacturing process of an individual or individualized orthosis or prosthesis.

If the control is carried out on the basis of a state machine in the orthopedic device, there is the possibility that an assessment of sensor data is carried out by the comparison to data stored in the computer network. If individual data or transitions of individual states do not occur at all in a specific user, the control can be modified in such a way that conditions which have to be fulfilled to trigger or block specific transitions are no longer queried. Those conditions which never occur or are never queried can then be omitted if they do not relate to safety-relevant states. The control thus becomes faster or more precise or consumes fewer resources. Omitting transitions or conditions can be carried out automatically in the background by a software update or by an updating service.

From the stored data during the use by a patient, together with the version history of the respective controller or the program and with the identified hardware components and the user profile, a proposal is developed for how the settings of the orthopedic devices are to be changed to be able to perform optimization and an adapted control to the respective user. Training programs or other items of education information can also be developed on the basis of the specific usage data and the user profile and transmitted to the user, the therapist or orthopedic technician, or the producer.

If the orthopedic device is not being used or is located in a charging station for cleaning or charging of rechargeable batteries, a data exchange and a separate analysis can take place automatically. For example, if it is detected that a component is defective, a wear limiting value is almost reached, or a maintenance deadline is imminent, corresponding information can be output to the user and/or a technician or therapist, so that independently of the occurrence of an error and without action by the user, a repair order or maintenance or also a software update can be initiated. An item of information can be given to the user that a corresponding order has been placed or corresponding maintenance or repair is necessary.

It is also possible to output an item of information to the user that and how he has used the orthopedic device. In the event of incorrect usage, suggestions for another way of use or suggestions to avoid overloads can be transmitted, for example, to the mobile terminal. The automatic information about the state of the orthopedic device can also be transmitted via the cloud or the computer network to the producer.

A further monitoring function can be carried out during registration and connection of the orthopedic device to the computer network in that the data are provided for analysis via the app and the mobile terminal. The analysis in the evaluation unit links a data pool made up of data of other orthopedic devices to the data of the specific orthopedic device and transmits the information to the orthopedic technician, the supplier, and/or the producer, so that they each have a status report of the orthopedic device. On the basis of the analysis and the data evaluation of all comparable orthopedic devices as well as the experiential values of the producer, it is possible to initiate steps for error avoidance, provide them via a transmitter to the user via the terminal and the app, and transfer them to the orthopedic device. It is thus also possible to define variable maintenance intervals for the entire orthopedic device or its components. The user can also approach the producer and/or orthopedist and/or orthopedic technician directly via the app if in his opinion something is wrong with the prosthesis or orthosis or the exoskeleton. He can then also transmit photos, videos, speech messages, etc. at the same time, which assist and facilitate the comprehension of the problem.

Both the mechanical components and also the electronic components and the software can be adapted in dependence on the type of use, usage duration, and usage intensity, so that excessive maintenance is avoided or, in the case of elevated load, the use of the orthopedic device in a state which would require maintenance is avoided. The orthopedic device thus recognizes when a service or a maintenance is necessary and initiates this as needed, so that a reduction of the expenditure results for the user, the orthopedic technician, or the producer.

As an assistance of a rehabilitation process or to maintain the physical capabilities, a rule set or a measure catalog of exercises to be carried out can be worked out or adapted for the respective user on the basis of the transmitted data. Prior exercises for maintaining or reestablishing physical functions can also be incorporated. The judgment of the prior exercises and the changes of the settings within the orthopedic device are adapted depending on the training on the basis of the achieved results. The judgment can be carried out either inside the orthopedic device, in the app, or on a server, for example within the computer network. The training is therefore adapted to the individual possibilities of the user and changed in dependence on the actual training effort and training progress. In combination with the training progress or the training state, settings of the orthosis or prosthesis or the other orthopedic device can then be changed, for example in the case of an artificial knee joint, the damping values for a flexion during a sitting down process can be reduced if training progress has been achieved or, vice versa, the damping can be increased if the physical capabilities have decreased.

The sensors for acquiring sensor data are in particular those sensors which are already provided and necessary for the controller of the function of the orthopedic device. If the data of these sensors alone do not yet permit an inference about states or changes or other desirable parameters, it can be necessary to define additional sensors or additional states and rule sets within the controller. The sensor data of the sensors which are absolutely necessary for the functionality of the orthopedic device are thus placed in a corresponding context in order to be able to infer the desired information content from the data.

If a risk assessment is present in the controller stored in or associated with the orthopedic device or in the control program, in which a certain hazardous situation is assumed upon an occurrence of a specific state or a specific combination of sensor values, an adaptation of the risk assessment can be carried out on the basis of the evaluation of the usage data in a plurality of such or comparable orthopedic devices. If a risk of a fall is assumed in the case of an assumed combination of sensor values in a specific situation in the original controller and a corresponding safety routine is activated and if it proves in a very large number of such situations or in all situations that there is actually no risk for the user, the risk assessment can be changed and if necessary the controller can be changed automatically or after a check by an orthopedic technician or the producer of the orthopedic device. A safety query can then be deleted or modified, whereby the controller is simplified as a whole, the response behavior is improved, and the control complexity can be reduced.

A further aspect of the invention provides that data of different sensors are used jointly and possibly in a weighted manner to obtain the desired items of information. For example, data on the spatial position, on forces, and on electromyographic signals can be arranged on the orthopedic device to recognize specific movement patterns. The different sensors record different variables and permit different inferences with respect to the respective situation with a differing accuracy. It can therefore be advantageous if multiple or all available sensor data are used to acquire a desired characteristic variable or to acquire, for example, a movement pattern, in order to evaluate whether or not this movement situation or load situation actually occurs. The various sensor values can be provided with a different weighting factor for the various situations. The analysis of an occurring force using a force sensor can be assumed to be accurate with a very high probability. Inferences about occurring forces on the basis of electromyographic sensor data are rather less accurate. A corresponding weighting or factorization of the individual sensor values in dependence on the items of information to be obtained and the association in a confidence probability is therefore reasonable. In addition, all sensor values which may be relevant for the acquisition of the respective characteristic variable can be combined with one another and ranked with a common confidence factor, so that the probability of the precise recognition of the parameters or the items of information is carried out on the basis of fused sensor data. On the basis of individual and fused sensor data in conjunction with the respective associated weighting, the final decision can then be made as to whether a situation exists, whether a specific variable is present, or an item of information can be assessed as accurate.

In sensors which are applied directly to the patient or user of the orthopedic device or which can acquire items of information about the user of the orthopedic device, an assessment can take place as to whether specific usage periods or types of use are connected in conjunction with the detected physical quality. For example, dermatological monitoring of the stump can be carried out in the case of a prosthesis or the limb in the case of an orthosis. If a prosthesis is equipped with a liner, the state of the skin, for example moisture, salt content in the sweat, fat content, or the like can be monitored by sensors in the liner. The sensors are connected to the transmitter and the evaluation unit in the computer network. If an overload or a dermatological overload is detected, a usage restriction can be recommended or a medical product or medication for treatment or prophylaxis against dermatological damage or other damage can be transmitted to the user. Therapy recommendations can also be output, for example for electroacupuncture or a treatment of phantom limb complaints or phantom perceptions or the like.

If the orthopedic device is to be set on the basis of sensor data, for example to change the setting of a prosthesis, changed parameters can be created and transmitted automatically. The possibility fundamentally exists that the changed parameters are automatically transmitted and implemented; to increase the level of safety, in the context of a video analysis or also on location with an orthopedic technician or another expert, the adjustment can be carried out during the use of the orthopedic device. If the expert or orthopedic technician is not on location, for example, the adjustment has to be started by the expert or the orthopedic technician during a remote treatment via a video telephone call or telephone call. The user can release the orthopedic device for maintenance by pressing a button on the orthopedic device or on the terminal, so that a remote update can only take place upon the release by the user in conjunction with a release by the orthopedic technician or expert.

Using the method, it is possible that experts or therapists establish a remote orthopedic practice via the Internet and the computer network in order to assist a local specialist, who does not have to be an expert, on location and/or to educate the user of the orthopedic device by video analysis, telephone conversation, and the data from the system. It is possible from the data in conjunction with items of acoustic information via telephone and optical information via video transmission to establish a location-independent orthopedic practice which creates algorithms with assistance of the data from the computer network in order to analyze movement sequences, give automated proposals for changes in the software, and/or perform adjustments to the hardware. The algorithms can be transmitted to the expert, who then relays them individually to the respective patient, adapts them, and assists the specialist on location.

Due to the coupling of the orthopedic device to a computer network, it is possible to place an automatic emergency call upon detection of an emergency situation, for example a fall or the like. The orthopedic device can be provided with a locating module, for example a GPS module, to transmit the location data together with the emergency call. If the terminal or the orthopedic device is equipped with a SIM card, a location determination of the user and thus also the location assignment of the emergency can take place simultaneously via the localization of the SIM card. In addition to an automatic recognition of an emergency situation, the orthopedic device and/or the mobile terminal can be equipped with an emergency button which instructs a suitable institution, for example an emergency service, an orthopedic technician, the producer, or the like about the problem. In addition to a fall, technical problems can also be automatically reported, so that, for example, in the event of decreasing capacity of the rechargeable battery, a replacement rechargeable battery can be ordered early and delivered or offered for exchange.

In addition to emergency situations, properties of the orthopedic devices can also be changed on the basis of the data of a locating function, such as the change of the volume of signals/feedback upon leaving a building or changing the movement modes in dependence on the environment.

If the users of the orthopedic device desire this, they can be compiled into groups and equipped with contact data among one another, so that a social group can form via the computer network, for example, to perform an experience exchange or to plan and carry out shared activities.

In principle, there is the option that additional services are also switched on separately and enabled individually for the respective user or orthopedic technician, possibly subject to cost, via the computer network in addition to a basic supply of software and items of information.

One refinement of the invention provides that additional items of information are provided via the tracking of the route on which the user of the orthosis or prosthesis or other orthopedic device moves. Via a route recognition, which can take place via a GPS system, the present way or route is acquired and compared to stored environmental data. The environmental data can comprise, for example, the surface profile of the covered way or route or also the expected way or route still to be covered, so that on the basis of the stored items of information and the assessment of which route is taken, changes can be performed in the orthopedic device. For example, transmission ratios can be changed, dampings can be changed, actuators can be activated differently, or possibly also route recommendations can be given if it is to be expected that a certain route cannot be managed or can only be managed with elevated risk. The orthopedic device can be adapted to the present terrain and terrain possibly to be expected. The energy management can be adapted accordingly. If a unit for converting kinetic energy into electrical energy is present in the orthopedic device and this conversion is therefore possible, the energy management system can perform a correspondingly changed energy regime in the event of a conversion to be expected, for example due to hill ascent or hill descent, for example can assess the available energy reserves differently.

One refinement of the invention provides that the usage data of orthopedic devices are collected and locations which are frequented often by users of the orthopedic devices are recognized and considered separately. A proposal adapted to the respective usage form can be distributed to the respective user by the analysis of the movement patterns of the users of the orthopedic devices. Thus, a beloved and interesting destination, for example a point of interest, can be recommended via a display unit, wherein an optimized path recommendation can be given using the recognized movement patterns of other orthopedic devices. One refinement of the invention provides that together with location data of various control situations and movement situations, a usage of an orthopedic device is recognized. If tripping processes are frequently recognized in a specific region or at a specific location, these locations or regions can be transmitted together with the GPS data to the respective controller. A warning message can be output upon reaching or approaching points which are linked to an increased hazard for the user of an orthopedic device. On the other hand, there is also the option that locations without reported incidents or disturbances of the normal movement sequence are designated as particularly worthwhile and suitable for the user of the respective orthopedic device and are recommended.

One refinement of the invention provides that a map app for different degrees of mobility including a GPS module is associated with the orthopedic device. Route finding would thus be possible in consideration of a path quality, for example paths without staircases or with a flat slope can be taken into consideration in the route selection. Depending on the degree of mobility, another route recommendation adapted to the respective user can be output. The covered route can simultaneously also be acquired as a movement pattern and stored, so that the degree of activity of the respective user is ascertained. Similarly to a fitness app, it can be ascertained whether and how the respective user of the orthopedic device moves. Based thereon, movement recommendations can be given. In addition, it is possible that the predicted movement duration or duration of a route is adapted in dependence on the individual movement data to the degrees of mobility of the respective user. The predicted or estimated time for covering a specific path can be adapted to the personal average speed of the respective user; it can possibly be taken into consideration that various obstacles are located on the path, so that the predicted route changes in dependence on the obstacles to be expected.

Qualities of paths can be entered in the map, for example staircases, obstacles, slopes, curbs, gravel paths, or asphalt. There is furthermore the possibility of the user making entries himself, for example identifying obstacles and proposing alternative routes. One refinement of the invention provides that an app is configured for the local public transport with assessment with respect to the respectively existing degrees of mobility. The route or travel planning via public transport can be carried out according to classification according to degrees of mobility in the case of orthoses or prostheses or the selected other orthopedic devices. Buses, streetcars, trains, railway stations, bus stops, and the like can be assessed, as well as how they are reached and how they are to be used with the respective different degrees of mobility or other orthopedic devices. One relevant aspect would be, for example, a flat entry, a wide entry, railings to hold onto, or the presence of elevators, escalators, or the like.

One refinement of the invention provides that an environmental recognition is configured, for example by a corresponding sensor system. Ultrasonic and/or radar sensors can scan the environment, the possibly provided actuators cause a change of the controller before a hazardous situation occurs. In orthoses and/or prostheses, ankle joints and knee joints can be activated jointly, so that adapted damper behavior can be produced upon recognition of an occurring hazard or an imminent hazard. Ultrasonic sensors, radar sensors, cameras, or sonar can be used. Sensors generate an environmental map, on the basis of which it can be recognized in which environment the user of the orthopedic device is moving. An assessment is carried out as to which obstacles are located in the environment or with which movement situations the user will presumably be confronted. For example, if a staircase is recognized, changed damper behavior or drive behavior can be activated. The quality of the ground can be recognized from image data or other sensor data, whereby, for example, an activation of an actuator to increase the dorsiflexion can result. The sensors can be arranged at every point of the orthopedic device; for example, in prosthesis devices in the respective knee joint, in the event of problems with respect to the housing, the sensor can also produce data via a knee-ankle coupling for a foot element, for example a prosthetic foot or a footplate of an orthosis.

One refinement of the invention provides that the orthopedic device, for example orthosis, prosthesis, or wheelchair, is individualized and design proposals are submitted via a data network, which can be assessed by other users of an orthopedic device, in particular a comparable orthopedic device, and can possibly be selected. Within a time period, a specific number of proposals and designs can be submitted or uploaded and released for adaptation. Various specifications can also be made, for example a theme, on the basis of which a new design proposal can be submitted. The theme can be changed depending on the season or on special occasions. By networking multiple users of an orthopedic device, it is possible for the respective users to refer to one another in the case of a problem, so that, for example, in the event of an empty energy accumulator or a missing charger, a user of an orthopedic device places an emergency call in the cloud or in the data network in order to obtain help from other users of a similar or comparable orthopedic device. An app can indicate users of a similar orthopedic device in the vicinity, possibly in anonymized form. It is also possible that the user of an orthopedic device can search for other users of an orthopedic device if they wish.

One refinement of the invention provides that first a base model of data and data service is provided, wherein additional services or features can be purchased or unlocked, for example in return for released data or user profiles.

Due to the reclamation of energy by movement, for example by walking or by hill descent, power can be stored in an energy accumulator, whereby the rechargeable battery running time may be extended.

New radio technologies can be integrated, for example LTE or 5G, to enhance the quality of the services. Systems which transmit data directly to a server can be integrated into the orthopedic device, so that later evaluations and automatic learning are facilitated.

The configuration of orthopedic devices can be carried out with the assistance of augmented reality, for example by the use of virtual reality glasses, hollow lenses, augmented reality glasses, or the like. Required data can be overlaid directly, the respective environment can be acquired, also measurement and object recognition can be carried out via the acquisition units, such as glasses or the like, so that an evaluation and measurement via cameras of the glasses can be carried out with a direct evaluation of data. An authentication element can be integrated into a prosthetic hand, for example a mass storage device, an RFID, a chip card, a Bluetooth connection, or the like, in order to be able to carry out an automatic authentication solely by touching or approaching the prosthetic hand on interfaces, such as ATMs, in the hospital, the Internet, or other access controls. This can also be carried out by the integration of the corresponding authentication unit in other orthopedic devices, for example prosthetic knee, prosthetic feet, prosthetic arms, or other orthopedic aids. A corresponding payment function can also be integrated.

Sport and rehabilitation exercises can be acquired. The sport mats or sports equipment are equipped with sensors, as are the orthopedic devices, so that movements are acquired. The sensor data are combined and evaluated to acquire who executes which exercises how. Improvement proposals, alternative exercises, exercise recommendations, and an acquisition of the improvement or possibly worsening of a state could be carried out.

The orthopedic device can be used, as an IoT hub, as a possible platform for additional systems or software, so that the prosthesis, orthosis, or the other orthopedic device can be expanded easily. New functions can be integrated, which possibly have nothing to do with the orthopedic device as such.

Exemplary embodiments of the invention are explained hereinafter on the basis of the appended figures. In the figures:

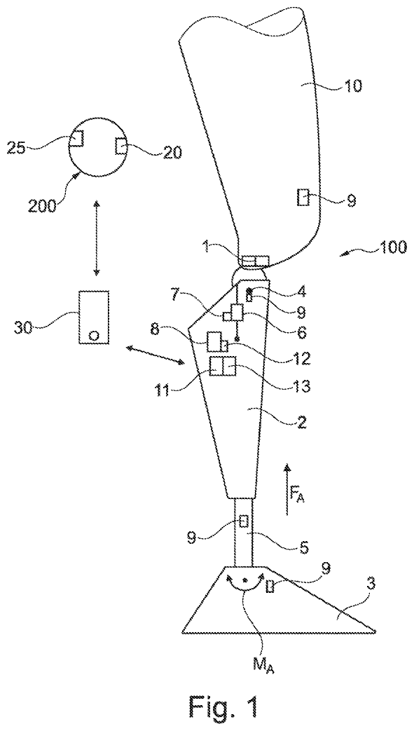

FIG. 1—shows a schematic illustration of an orthopedic device;

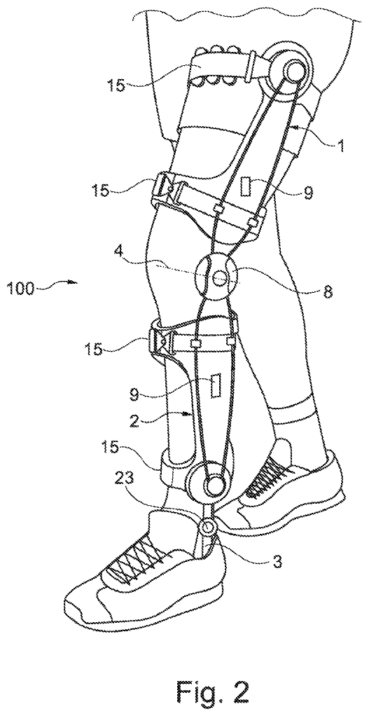

FIG. 2—shows a variant of the invention with an orthosis; and

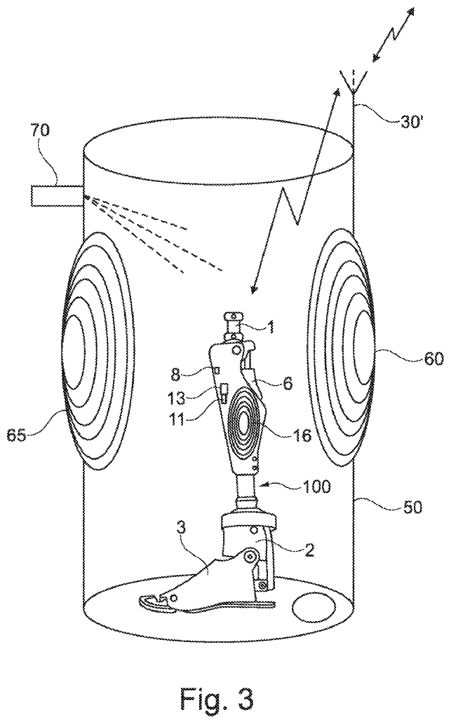

FIG. 3—shows a variant of the invention in a storage system.

FIG. 1 shows a schematic illustration of an orthopedic device 100 in the form of a leg prosthesis having a prosthetic knee joint having an upper part 1, on which a thigh socket 10 is fastened for receiving a thigh stump of a patient. A lower part 2 in the form of a lower leg part is pivotably mounted around a pivot axis 4 on the upper part 1. The lower part 2 has a lower leg tube 5, on the distal end of which a prosthetic foot 3 is fastened; the prosthetic foot 3 can be pivotably mounted on the lower leg tube 5.

A plurality of sensors 9 are arranged in the orthopedic device 100 in order to collect sensor data with respect to the usage or the state of the orthopedic device 100. In the illustrated exemplary embodiment, for example, a sensor 9 is arranged in the foot part 3 for acquiring an ankle torque M_{A}. A sensor 9 for acquiring an active axial force F_{A} is arranged in the lower leg tube 5, for example, an angle sensor 9 is fastened on the lower part 2, possibly in combination with an inertial angle sensor for acquiring the spatial location of the lower part 2 during the use of the orthopedic device 100. Inertial sensors 9, torque sensors, angle sensors, temperature sensors, and other sensors can be arranged or integrated in the thigh socket 10.

Furthermore, a resistance unit 6 in the form of a damper or an actuator is arranged in the lower part 2, which is supported between the upper part 1 and the lower part 2 in order to provide a settable extension resistance and flexion resistance, in particular when the resistance unit 6 is designed as a passive damper. The resistance unit 6 is associated with an adjustment unit 7, for example a motor or another drive, a magnet, or the like, via which the respective resistance within the resistance unit 6 is changeable. If the resistance unit 6 is designed as a hydraulic damper or pneumatic damper, the adjustment unit 7 can increase or decrease the respective flow cross section of an overflow channel between an extension chamber and a flexion chamber or change the flow resistance in another way. It is also possible that the adjustment unit 7 changes the viscosity of the fluid located inside the resistance unit 6, for example by changing a magnetic field and acting on a magnetorheological liquid. If the resistance unit 6 is designed as an electric motor, it can be operated in the generator mode, whereby a change of the mechanical resistance against a flexion or extension of the upper part 1 relative to the lower part 2 can take place. If the resistance unit 6 is designed as a motor and is operated in the motor mode, active assistance of the flexion and/or extension of the prosthetic device is possible.

The adjustment unit 7 is coupled to a control unit 8 in order to be able to perform an activation or deactivation of the adjustment unit 7, in order to influence the flexion and/or extension, and possibly to block the joint. The desired behavior of the orthopedic device 100 is set via a corresponding activation signal or deactivation signal to the adjustment unit 7 via the control unit 8. The adjustment unit 7 is activated or deactivated via the control unit 8 on the basis of sensor data which are transmitted from the sensors 9 to the control unit 8. The sensor data sensors 9 can be preprocessed or processed in the control unit 8 itself, for example to calculate angular velocities or accelerations from angle data. If the desired data are ascertained directly by the sensors, for example via angle sensors, acceleration sensors, or the like, a corresponding preparation can be omitted. The control unit 8 is equipped with or coupled to computers or processors in order to evaluate the data electrically or electronically and to carry out a corresponding activation or deactivation of the adjustment unit 7 on the basis of this evaluation. The sensors 9 are connected to the control unit wirelessly or via wire or by other line units. If a wireless communication takes place between the sensors 9 and the control unit 8, corresponding transmitter and receiver units are provided within the control unit 8 and the sensors 9 or associated with them.

The control unit 8 can also include a storage unit 12 or can be coupled to such a storage unit 12, which records sensor data and sensor data curves or evaluations of sensor data and sensor data curves to be used further or transmitted at a later time.

The control unit 8 is also associated with a transmitter 11 and a receiver 13 to transfer sensor data, programs, access rights, settings, changes of settings, software updates, or other things from external units to the orthopedic device.

A data acquisition is performed via the sensors 9 during the use of the orthopedic device 100, in which all relevant data such as forces, torques, accelerations, orientations, temperatures, deformations, movement time periods, usage time periods, and the like are acquired and transferred to the control unit 8 and possibly stored. Sensor data and the like are transferred from the control unit 8 and the storage unit 12 via the transmitter 11 either directly to an evaluation unit 20 in a computer network 200, if the transmitter 11 is configured to communicate directly with a computer network 200 or the evaluation unit 20. For this purpose, a SIM card for establishing a telecommunication connection or another communication interface for establishing a connection to a transmitter network or the like can be provided in the orthopedic device 100. Alternatively or additionally, the transmitter 11 can transmit the data via a mobile terminal 30, for example a smart watch, a tablet, a mobile telephone, or a laptop, which are transmitted from there out of the computer network 200. The evaluation unit 20 is arranged or formed in a centralized or decentralized manner in the computer network 200 and is possibly coupled to a database 25 in order to store the sensor data or the evaluation thereof as well as other data related to the use of the orthopedic device 100.

An evaluation, possibly a risk analysis having recommendations, software updates, and the like, is carried out on the basis of the sensor data and the other data in the evaluation unit 20 or in the computer network 200.

Questions on the subjective judgment of the behavior of the orthopedic device 100 can also be stored in the mobile terminal 30 or transferred from the computer network 200 therein in order to place questions adapted to the usage behavior and obtain feedback of the respective user without an orthopedic technician interposed. Units for position determination can be integrated both in the orthopedic device 100 and also in the mobile terminal 30, for example a GPS module or another location determination unit, which carry out a corresponding localization, for example, via transmitter cells in mobile radio systems. An improved evaluation of state data or changes which are acquired by the sensors 9 can be carried out on the basis of the position data.

FIG. 2 shows an alternative orthopedic device 100 in the form of a knee-ankle orthosis, having an upper part 1 having an adjustable thigh rail, a lower part 2 having a settable lower leg rail, and a foot part 3 which is pivotably mounted in an articulated manner around an ankle joint axis 23. The upper part 1 is coupled to the lower part 2 pivotably around a joint axis 4. Sensors 9, which can be designed similarly to the orthopedic device 100 according to FIG. 1, are shown both on the upper part 1 and also on the lower part 2. In the region of the knee joint axis 4, the control unit 8 is arranged; other positionings are also possible. All units of the orthopedic device 100 of FIG. 1 are shown on the orthopedic device 100 according to FIG. 2, but can also be or become arranged accordingly there. The transfer of sensor data also takes place here via a transmitter 11 (not shown) to the computer network 200 for evaluation in the evaluation unit 20.

A variant of the invention is shown in FIG. 3, in which the orthopedic device 100 is shown in a non-attached state. The thigh socket 10 is removed from the upper part 1, the individual components of FIG. 1 are only partially shown. In addition to the components according to FIG. 1, a supply connection 16 is arranged in the lower part 2 of the orthopedic device 100, which is designed as a wireless supply connection 16 in order to transfer energy wirelessly in the idle state of the orthopedic device 100. The supply connection 16 includes coils, for example, which are excited via a corresponding supply unit 60, which can be housed on an outer wall of a holder 50 for storing the orthopedic component 100 in the removed state, in order to transfer energy inductively. The orthopedic device 100 can therefore be placed in the holder 50 for maintenance purposes, in order to ensure the electrical supply. A cleaning arrangement 70 can be arranged on the holder 50 to be able to perform a disinfection or similar cleaning. An identification unit 65 can also be arranged on the holder 50, via which a code, for example an optical code or an electromagnetic code, can be read to identify the orthopedic device. Moreover, a transfer module 30′ is arranged on the holder 50, which includes a transmitter and a receiver. The transfer module 30′ can receive data from the transmitter 11 of the orthopedic device 100 and transmit data to the receiver 13. The transfer module 30′ can simultaneously receive data from the evaluation unit (not shown) of the computer network 200, which is equipped with a transmitter and a receiver, or transmit data to such a computer network 200. Therefore, only a low transmission power of the transmitter 11 to the transfer module 30′ is required, whereby energy can be saved. The data transfer preferably takes place in the removed state of the orthopedic device 100, for example at night, when computer power is not required for processing sensor signals for controlling the extension and flexion or other functions. Moreover, in the removed state with the arrangement in a holder 50 and an energy supply, there is the option of extending the usage duration of the orthopedic device 100, since less energy is used for the evaluation and data transfer.

1. A method for evaluating usage data of at least one orthopedic device, which is equipped with sensors for acquiring properties, states, or changes of properties or states, wherein the sensors are connected to a transmitter directly or via a storage unit and the transmitter transmits the sensor data provided by the sensors to an evaluation unit in a computer network, in which the sensor data are processed.

2. The method as claimed in claim 1, characterized in that movement data, operating data, and/or loads of the orthopedic device or sensor data about the user of the orthopedic device are acquired by the sensors and transmitted to the evaluation unit.

3. The method as claimed in claim 1, characterized in that the sensor data are acquired and evaluated in a chronologically limited manner or over the entire usage time period or usage time periods of the at least one orthopedic device.

4. The method as claimed in claim 1, characterized in that the sensor data are supplemented with assessments of the user of the orthopedic device and/or third parties.

5. The method as claimed in claim 4, characterized in that the assessments are carried out on the basis of provided questions.

6. The method as claimed in claim 1, characterized in that the sensor data are transmitted in real time, during usage pauses, at defined times, on request of the evaluation unit, and/or by transmission by the user to the evaluation unit.

7. The method as claimed in claim 1, characterized in that the sensor data are coded in a personalized and/or device-specific manner.

8. The method as claimed claim 1, characterized in that sensor data of various similar or comparable orthopedic devices are compiled and evaluated in the evaluation unit.

9. The method as claimed in claim 1, characterized in that sensor data of comparable users or sensor data of multiple users combined to form a group are acquired and evaluated in the evaluation unit.

10. The method as claimed in claim 1, characterized in that a risk analysis is carried out on the basis of the sensor data and a recommendation is output to the user, a payer, an orthopedic technician, and/or a producer of the orthopedic device on the basis of the results of the risk analysis.

11. The method as claimed in claim 1, characterized in that usage instructions, warning messages, and/or requests are communicated to the user on the basis of the evaluation.

12. The method as claimed in claim 1, characterized in that a control unit, which is coupled to a receiver, via which control data, access rights, sensor calibrations, and/or the changes thereof and/or recordings about changes are transmitted, is associated with the orthopedic device.

13. The method as claimed in claim 1, characterized in that settings, changes of settings, calibrations, accesses to the sensor data, and access rights are stored and kept retrievable in the computer network.

14. The method as claimed in claim 1, characterized in that multiple orthopedic devices are coupled with one another and exchange data via the computer network.

15. The method as claimed in claim 1, characterized in that data are transmitted to the computer network via a mobile terminal.

16. The method as claimed in claim 1, characterized in that summaries of selected parameters which are acquired by the sensors or are derivable from the sensor data are transmitted to the user of the orthopedic device, a payer, an orthopedic technician, and/or a producer of the orthopedic device.

17. The method as claimed in claim 16, characterized in that a parameter curve is ascertained and displayed.

18. The method as claimed in claim 1, characterized in that a specific data structure is created for each user and/or for each orthopedic device, on the basis of which the sensor data are selected and transmitted to the evaluation unit.

19. The method as claimed in claim 1, characterized in that a selection from multiple alternative orthopedic devices is made on the basis of the evaluation and proposed to the user, orthopedic technician, and/or payer.

20. The method as claimed in claim 1, characterized in that the evaluation unit correlates data about the user of the orthopedic device and/or the orthopedic device and/or comparable orthopedic devices and derives therefrom probabilities about malfunctions or disadvantageous effects and transmits these probabilities to the user, the orthopedic technician, the payer, and/or the producer.

21. The method as claimed in claim 1, characterized in that the sensor data and/or evaluated sensor data are stored in a database.

22. The method as claimed in claim 1, characterized in that the access to the evaluation unit and/or the computer network is restricted.

23. The method as claimed in claim 1, characterized in that an orthosis, exoskeleton, or prosthesis is used as the orthopedic device and the sensor data are acquired during the use of the orthosis, the exoskeleton, or the prosthesis.
